(19) United States
(12) Patent Application Publication
(10) Pub. No.: US 2026/0104425 A1
(43) Pub. Date: Apr. 16, 2026

(54) AN ANALYTICAL METHOD USING LC-MS/MS PROTEOMICS TO CHARACTERIZE PROTEINS TRANSLATED FROM MRNA

(71) Applicant: Merck Sharp & Dohme LLC, Rahway, NJ (US)

(72) Inventors: Xuanwen Li, Belle Mead, NJ (US); Alyssa Quencer Stiving, Philadelphia, PA (US); Mark Haverick, Whitehouse Station, NJ (US); Benjamin William Roose, Philadelphia, PA (US); Christopher Michael Tubbs, Hatfield, PA (US)

(73) Assignee: Merck Sharp & Dohme LLC, Rahway, NJ (US)

(21) Appl. No.: 19/116,729

(22) PCT Filed: Sep. 28, 2023
(86) PCT No.: PCT/US2023/033931

§ 371 Date: Mar. 28, 2025

Related U.S. Application Data
(60) Provisional application No. 63/411,948, filed on Sep. 30, 2022.
(60) Provisional application No. 63/500,137, filed on May 4, 2023.

(51) Int. Cl. G01N 33/68; C12N 15/67; C12N 15/88; C12Q 1/37; G01N 30/02; G01N 30/06; G01N 30/72; G01N 30/88

(52) U.S. Cl. CPC G01N 33/6848; C12N 15/67; C12N 15/88; C12Q 1/37; G01N 30/7233; G01N 2030/027; G01N 2030/067; G01N 2030/8831; G01N 2333/96411; G01N 2333/96441

(57) ABSTRACT

The present disclosure provides a method of assessing translation efficacy of an mRNA using liquid chromatography-tandem mass spectrometry (LC-MS/MS). The mRNA is first translated into protein either in a cell lysate (cell-free translation; CFT) or inside a cell (cell-based translation; CBT) and analyzed using LC-MS/MS. The method provides advantages such as speed and convenience over traditional immunoassay-based methods of detecting translated proteins. Translation using CBT may be necessary in certain formulations of the mRNA, such as when the mRNA or a mixture of mRNAs is encapsulated inside a lipid nanoparticle.

### FIELD OF THE INVENTION

The invention relates to methods for assessing translation efficacy of an mRNA using cell-free translation (CFT) or cell-based translation (CBT), followed by liquid chromatography-tandem mass spectrometry.

### CROSS-REFERENCE TO RELATED APPLICATIONS

This application claims the benefit of U.S. Provisional Patent Application No. 63/411,948, filed Sep. 30, 2022, and U.S. Provisional Patent Application No. 63/500,137 filed May 4, 2023, the disclosure of which is incorporated herein by its entirety.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Jan. 30, 2023, is named 25583-WO-PCT_SL.XML and is 295,965 bytes in size.

### BACKGROUND OF THE INVENTION

Messenger RNA (mRNA) is acquiring increasing importance as a new class of therapeutics in which genetic information is delivered to the cells (Sahin U et al., Nature Reviews Drug Discovery, vol 13, October 2014, 759-780). Once the mRNA is delivered, cellular machinery translates the mRNA into a functional protein. In the field of vaccines, mRNA-based therapeutics are of great interest as being safe and efficient alternatives to traditional live virus or protein-based vaccines (Kramps, T., Probst, L, RNA 2013, 4(6) 737-749). In mRNA-based vaccines, the mRNA encodes a viral protein that generates a protective immune response. mRNA-based therapeutics are of interest in replacement therapeutics as well. In these therapeutics, the mRNA encodes an absent or dysfunctional protein that, when functionally expressed in the patient, either supplies the missing protein or compensates for the dysfunctional protein (Damase T R et al., Frontiers in Bioengineering and Biotechnology, vol. 9, March 2021, 1-24).

Success of mRNA-based therapeutics depends on several factors including, translational efficacy of the mRNA, its delivery, immunogenicity, and ease of manufacture. With respect to translational efficacy, it is well known that RNA is not stable and can undergo degradation at different stages, for example, during preparation, formulation, and storage, thereby impacting translation efficacy. Translation efficacy may also be affected at the translation initiation stage, for example, because of the 5′-UTR used. Additionally, formulation of the mRNA, such as in lipid nanoparticles (LNPs), may also influence translation of the mRNA.

Thus, there is a need for reliable analytical methods for characterizing mRNA translation efficacy.

### SUMMARY OF THE INVENTION

Described herein is a method to assess translation efficacy of an mRNA using liquid chromatography-tandem mass spectrometry (LC-MS/MS). The mRNA is first translated into a protein either in a cell lysate (cell-free translation (CFT), also referred to herein as in vitro translation (IVT)) or inside a cell (cell-based translation (CBT)) prior to analysis by LC-MS/MS. The method (also referred to as CFT-MS or CBT-MS) provides certain advantages over traditional immunoassay-based methods of detecting translated proteins since it does not require an antibody specific to the translated protein.

Accordingly, in one aspect, provided herein is a method for assessing translation efficacy of one or more mRNAs. The method includes the following steps:
(a) translating the one or more mRNAs, wherein the one or more mRNAs are present in a cell lysate (CFT) or within a cell (CBT), thereby obtaining a translation reaction product comprising a protein or proteins translated from the one or more mRNAs;
(b) digesting the translation reaction product with a first proteolytic enzyme, thereby obtaining a first enzymatic digest;
(c) subjecting the first enzymatic digest to liquid chromatography-tandem mass spectrometry (LC-MS/MS), thereby obtaining a first peptide profile; and
(d) analyzing the first peptide profile by detecting peptides derived from the one or more proteins; and
(e) optionally further analyzing the first peptide profile, thereby determining amino acid sequence or sequences of the one or more proteins; and comparing the amino acid sequence or sequences thus obtained to an amino acid sequence or sequences expected from translation of the one or more mRNAs, thereby verifying, nucleotide sequence or sequences of the one or more mRNAs;
   thereby assessing the translation efficacy of the one or more mRNAs.

In some embodiments, the method further includes the following steps:
(f) digesting the translation reaction product with a second proteolytic enzyme, thereby obtaining a second enzymatic digest;
(g) subjecting the second enzymatic digest to LC-MS/MS, thereby obtaining a second peptide profile;
(h) analyzing the second peptide profile by detecting and quantifying peptides derived from the one or more proteins;
(i) further analyzing the second peptide profile, thereby determining amino acid sequence or sequences of the one or more proteins; and comparing the amino acid sequence or sequences thus obtained to an amino acid sequence or sequences expected from translation of the one or more mRNAs, thereby verifying nucleotide sequence or sequences of the one or more mRNAs; and
(j) combining information from the amino acid sequence or sequences of the one or more proteins obtained in step (e) with that obtained in step (i), thereby assembling an amino acid sequence or amino acid sequences of the one or more proteins; and comparing the assembled amino acid sequence or sequences to an amino acid sequence or sequences expected from translation of the one or more mRNAs, thereby further verifying nucleotide sequence or sequences of the one or more mRNAs; thereby assessing the translation efficacy of the one or more mRNAs.

The method may include one or more additional features described below in the form of different embodiments.

In some embodiments, translation is carried out in a cell lysate.

In some embodiments, translation is carried out within a cell. For example, the one or more mRNAs are provided in a lipid nanoparticle.

In some embodiments, analyzing in step (d) further comprises quantifying peptides derived from the one or more proteins.

In some embodiments, analyzing in step (h) further comprises quantifying peptides derived from the one or more proteins.

In some embodiments, assessing translation efficacy of the one or more mRNAs comprises comparing the translation of the one or more mRNAs to the translation of a control mRNA previously determined to have optimal translation. The control mRNA may be included in the cell-free or cell-based translation performed in step (a) and translated along with the one or more mRNAs. Alternatively, the control mRNA may be translated separately using a method according to steps (a)-(d), and optionally according to steps (a) and (f)-(h). Without limitations, the control mRNA serves as a control for the cell-free or cell-based translation reaction, i.e., informs whether the cell-free translation system or the cells being used for translation are functioning as expected.

In some embodiments, analyzing translation of the one or more mRNAs comprises identifying at least two peptides predicted to be generated by the enzymatic digestion of the one or more proteins translated from the one or more mRNAs.

In some embodiments, the MS1 intensity of a peptide in the peptide profile is indicative of the relative abundance of a translated protein from which the peptide is generated.

In some embodiments, prior to step (b), the translation reaction product is enriched for proteins by removal of non-protein components using a suspension trapping column, thereby yielding a protein-enriched translation reaction product. In related embodiments, step (b) comprises digesting the protein-enriched translation reaction product on the column and, prior to step (c), eluting from the column, peptides resulting from the digestion. These eluted peptides are used in step (c).

In some embodiments, prior to step (f), the translation reaction product is enriched for proteins by removal of non-protein components using a suspension trapping column, thereby yielding a protein-enriched translation reaction product. In related embodiments, step (f) comprises digesting the protein-enriched translation reaction product on the column and, prior to step (g), eluting from the column, peptides resulting from the digestion. These eluted peptides are used in step (g).

In some embodiments, the method further comprises enriching the first and/or second enzymatic digest for peptides using a C18 column prior to step (c) and optionally prior to step (g).

In some embodiments, the LC-MS/MS comprises separating peptides present in the enzymatic digest using reversed-phase liquid chromatography prior to performing mass spectrometry.

In some embodiments, the translation reaction product is treated to reduce viscosity prior to enzymatic digestion.

In some embodiments, the translation product is sonicated to reduce viscosity.

In some embodiments, the first proteolytic enzyme used for digesting the translation reaction product is selected from the group consisting of trypsin, chymotrypsin, Asp-N, Glu-C, Lys-C, Lys-N, elastase, thermolysin, proteinase K, Staphylococcus aureus v8 protease, and pepsin.

In some embodiments the second proteolytic enzyme used for digesting the translation reaction product is selected from the group consisting of trypsin, chymotrypsin, Asp-N, Glu-C. Lys-C, Lys-N, elastase, thermolysin, proteinase K, Staphylococcus aureus v8 protease, and pepsin.

In some embodiments, both the first and the second proteolytic enzymes used for digesting the translation reaction product are selected from the group consisting of trypsin, chymotrypsin, Asp-N, Glu-C, Lys-C, Lys-N, elastase, thermolysin, proteinase K, Staphylococcus aureus v8 protease, and pepsin.

In some embodiments, the first proteolytic enzyme is one of trypsin and chymotrypsin and the second proteolytic enzyme is the other of trypsin and chymotrypsin.

In some embodiments, the first proteolytic enzyme is one of trypsin and Lys-C and the second proteolytic enzyme is the other of trypsin and Lys-C.

In some embodiments, the cell-free translation is performed using rabbit reticulocyte lysate.

In some embodiments, the cell-free translation is performed using wheat germ extract.

In some embodiments, at least one known protein is added to the translation product prior to the enzymatic digestion to serve as a reference protein in step (d) and optionally in step (h). Without limitations, the at least one known protein serves as a control for the method from the enzymatic digestion step through the LC-MS/MS analysis step, including quantification of the translated protein.

In some embodiments, the at least one known protein comprises a protein selected from the group consisting of alcohol dehydrogenase, myoglobin, carbonic anhydrase, phosphorylase B, and protein A.

### BRIEF DESCRIPTION OF THE DRAWINGS

FIG. 1A is a schematic diagram showing cell-free translation (CFT) of mRNA and detection of the translated product by Simple Western™ (SW) or mass spectrometry.

FIG. 1B is a schematic diagram showing sample preparation workflow following in vitro translation of mRNA including analysis by LC-MS/MS. Stars indicate steps introduced to reduce viscosity of the CFT sample and otherwise facilitate analysis by LC-MS/MS.

FIG. 2A is a graph showing the number of protein groups identified in each of five separate cell-free translation reactions that were carried out with different mRNA constructs or a control with no mRNA, using wheat germ extract. From left to right, the reactions are (1) mRNA construct 1 (amino acid sequence 1). (2) mRNA construct 2 (amino acid sequence 2), (3) mRNA construct 3 (amino acid sequence 2). (4) Water (control, with no mRNA), and (5) firefly luciferase mRNA construct. mRNA constructs 2 and 3 use different codons but produce proteins having the same amino acid sequence. For LC-MS/MS analysis, raw data was searched against a wheat proteome database to which the sequence of the protein corresponding to the mRNA being translated in that translation reaction was added.

FIG. 2B is a graph showing abundance of the protein translated from the mRNA of interest (see FIG. 2A) as % of the total protein present in the CFT sample.

FIG. 2C is a graph showing MS1 intensity of translated proteins observed in each of the five cell-free translation reaction samples described in FIG. 2A. In each sample (except blank), an MS1 peak was found that corresponded to the peak expected from the protein translated in that reaction sample and none other.

FIG. 2D is a Table showing whether proteins translated from the various mRNA constructs could be detected by SW assay and MS. FIG. 2D also shows the MS1 peak areas corresponding to the constructs.

FIG. 3 shows an amino acid sequence coverage map of firefly luciferase generated from cell-free translation of firefly luciferase mRNA followed by LC-MS/MS analysis. Two separate CFT and LC-MS/MS analyses were performed, one using chymotrypsin and the other using trypsin. Sequence coverage with chymotrypsin, trypsin, and overall sequence coverage are shown. Only the “mass and MS/MS” (MS2) peptides with coverage are underlined. The overall sequence coverage (chymotrypsin and trypsin coverages) obtained by MS2 (mass and MS/MS) alone was 94.4% whereas, with MS1 (Mass only)+MS2, the overall sequence coverage of 100% could be obtained. Thus, with MS1 and MS2 analyses combined, and with the use of two enzymes, the identity and proper translation of the mRNA could be confirmed by CFT-MS. The Figure also discloses the amino acid sequence of firefly luciferase (SEQ ID NO: 1).

FIG. 4 is a graph showing cell-free translation of an mRNA encoding a test protein (Protein X). The mRNA was thermally degraded by incubating at 50° C. for different time periods. Integrity of the mRNA was determined by capillary electrophoresis (CE) analysis of the intact mRNA. As expected, a decrease in mRNA integrity was observed (filled circles). Following translation, Protein X was detected using either SW (filled triangles) or mass spectrometry (filled squares). The graph shows that the CFT-MS assay can be used to detect mRNA degradation and can be more sensitive compared to SW.

FIG. 5A is a diagram showing alignment of the amino acid sequences of two test proteins, X and Y that share 97.24% sequence identity.

FIG. 5B is a set of three graphs showing results from SW analysis of Protein X and Protein Y (see FIG. 5A). Results obtained using an antibody detecting both Proteins X and Y is shown first, that obtained using an antibody detecting only Protein X is shown second, and that obtained using an antibody detecting only Protein Y is shown third. The anti-Protein X antibody exhibited non-specific binding (see arrow mark) and the anti-Protein Y antibody weakly recognized protein X as well, i.e., exhibited cross reactivity (see arrow mark). Note that in the second panel, which shows results using antibody detecting only Protein X, the line for “Blank” overlaps that for Protein Y. The cross-reactivity of these antibodies is likely due to the high sequence identity between these two proteins.

FIG. 6A is a schematic diagram of an experiment in which mRNAs for two test proteins X and Y are mixed in different ratios and in vitro translation reactions are carried out with the mixtures obtained. The goal of the experiment was to test the reliability of CFT-MS to accurately determine relative abundances of all proteins translated from a mixture of mRNAs.

FIG. 6B is a graph showing relative abundances (%) of X and Y proteins in the various mixtures (see FIG. 6A) as determined by MS (summed MS1 intensity, as obtained from Proteome Discoverer search).

FIG. 7 is a graph showing MS1 intensity of proteins translated in Huh7 cells from a mRNA drug product comprising a mixture of six mRNAs, formulated as a lipid nanoparticle. The mRNAs are numbered 1 to 6. The dark and light bars correspond to loadings of 6.7 ng and 4.4 ng, respectively, of each mRNA.

### DETAILED DESCRIPTION OF THE INVENTION

Methods for assessing translation efficacy of an mRNA using cell-free or cell-based translation of an mRNA followed by liquid chromatography-tandem mass spectrometry (CFT-MS and CBT-MS, respectively) are disclosed herein, and the advantages such methods offer over the state of the art will be understood more readily by reference to the following detailed description of the various embodiments and examples.

An analytical assay for reliably assessing mRNA translation efficacy is important because while mRNA-based drugs are being explored intensely, RNA is not stable and can undergo degradation, thereby negatively impacting its translation into protein, and ultimately its efficacy. The CFT-MS and CBT-MS methods described herein provide such assays.

Unless defined otherwise, all technical and scientific terms used herein have the same meaning as commonly understood by one of ordinary skill in the art to which the disclosure pertains. In describing the embodiments and in the claims, certain terminology will be used in accordance with the definitions set out below.

As used herein, the singular forms “a”, “an”, and “the” include plural references unless indicated otherwise. Thus, for example, references to “the method” includes one or more methods, and/or steps of the type described herein and/or which will become apparent to one of ordinary skill in the art upon reading this disclosure.

As used herein, the term “about”, when modifying the quantity, such as a stated concentration range, time frame, molecular weight, temperature, or pH, refers to variation in the numerical quantity that can occur, for example, through typical measuring, handling and sampling procedures involved in the preparation, characterization and/or use of the substance or composition; through differences in the manufacture, source, or purity of the ingredients employed to make or use the compositions or carry out the procedures; and the like. Sometimes, such a range can be within the experimental error typical of standard methods used for the measurement and/or determination of a given value or range. The allowable variation encompassed by the term “about” will depend upon the system under study and can be readily appreciated by one of ordinary skill in the art. In certain embodiments, “about” can mean a variation of ±0.1%, 0.5%, 1%, 2%, 3%, 4%, 5% or 10%. Whenever a range is recited within this application, every whole number integer within the range is also contemplated as an embodiment of the disclosure.

It is noted that in this disclosure, terms such as “comprises,” “comprised,” “comprising,” “contains,” “containing” and the like are used in an inclusive sense, e.g., they can mean “includes,” “included,” “including” and the like. Such terms refer to the inclusion of particular ingredients or set of ingredients without excluding any other ingredients.

Terms such as “consisting essentially of” and “consists essentially of”, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound, i.e., they exclude additional unrecited ingredients or steps that detract from the novel or basic characteristics of the disclosure. The terms “consists of” and “consisting of” are closed ended. Accordingly, these terms refer to the inclusion of a particular ingredient or set of ingredients and the exclusion of all other ingredients.

“mRNA” or “Messenger RNA” as used herein refers to a nucleotide polymer comprising predominantly ribonucleotides and encoding a polypeptide or protein. mRNA typically comprises from 5′ to 3, a cap, an untranslated region, an open reading frame encoding a protein or polypeptide, a 3 untranslated region and a 3′ poly(a) tail. In some embodiments, the mRNA may comprise one or more modified or non-natural nucleotide residues.

The terms “in vitro translation” and “cell-free translation” may be used interchangeably in the present specification and refers to a cell-free method of synthesis of proteins.

“Lipid nanoparticle” (LNP), as used herein, refers to a system made of multiple components consisting typically of a phospholipid, cholesterol, an ionizable lipid, and a polyethylene glycol (PEG)-lipid. Unlike liposomes, which include one or more rings of lipid bilayer surrounding an aqueous pocket. LNPs typically assume a micelle-like structure, encapsulating drug molecules in a non-aqueous core. The cationic lipid helps electrostatically condense the negatively charged RNA into nanoparticles whereas the ionizable lipid, which is positively charged at the acidic pH of the endosome, is thought to enhance endosomal escape (Nigel Davies et al., Molecular Therapy: Nucleic Acids, 2021. Vol. 24, pages 36-384). Phospholipid and cholesterol contribute to structural stability of the LNP among other properties, and PEG lipid helps avoid renal clearance as well as enhance stability and decrease the aggregation of LNP, thereby increasing circulation time in blood (Han Na Jung et al. Theranostics 2022. Vol. 12, Issue 17, pages 7509-7531).

As applied to a polypeptide sequence, the term “% sequence identity” or “% identity” refers to the percentage of matching residues between at least two polypeptide sequences aligned using a standardized algorithm. Methods for aligning polypeptide sequences are known. Percent identity may be measured over the entire length of a polypeptide sequence or over a shorter sequence length.

In some experiments of the present disclosure, test proteins have been used, and they have been identified as Protein X and/or Protein Y (see Examples 4 and 5). Also, in some experiments, certain mRNAs have been used, and they have been identified as numbered mRNA constructs, with associated numbered amino acid sequences (see Example 3). It should be noted that from the context of these experiments, one of ordinary skill in the art would know how these proteins or mRNA constructs function in these experiments, and therefore, their true identity is not essential for an understanding of the results of these experiments.

Cell-Free and Cell-Based mRNA Translation Liquid Chromatography-Tandem Mass Spectrometry (LC-MS/MS)

In cell-free (or in vitro) translation, an mRNA of interest is translated into the protein encoded by the mRNA using a suitable cell-free translation system. Rabbit reticulocyte lysate and wheat germ extract are the two most frequently used systems for cell-free translation. In cell-based translation, the mRNA of interest is translated inside a cell. Cell-based translation is suitable when mRNA or a mixture of mRNAs is formulated to enter a cell such as when the mRNA or the mixture of mRNAs is encapsulated inside an LNP. In the methods descried herein, following translation (cell-free or cell-based), the protein(s) produced are detected and quantitated using LC-MS/MS. This method provides a measure of efficacy of the translation of the mRNA, which in turn is indicative of the quality of mRNA. Translated mRNA can be detected also using traditional immunoassay-based techniques, such as Simple Western™ (SW), which is an automated capillary-based Western Blot technique (Nature Methods volume 8, pages v-vi (2011)), or immunofluorescence for cell-based assay.

However, instead of an immunoassay-based technique, bottom-up LC-MS/MS was used herein to detect the translated protein(s) as well as to determine its abundance because of the advantages of using the latter. These advantages will become apparent from the following description.

Protein translated from an mRNA using a cell-free or cell-based translation system is necessarily present in a complex mixture containing relatively large number and quantity of proteins originating from the translation system itself (e.g., 30-50 mg/mL of endogenous protein is present in wheat germ extract). In part because of the complexity of the translation system mixture and the fact that the translated protein constitutes a tiny fraction of the total protein content, inventors of the present application utilized a bottom-up proteomics approach for the detection and quantitation of the translated protein.

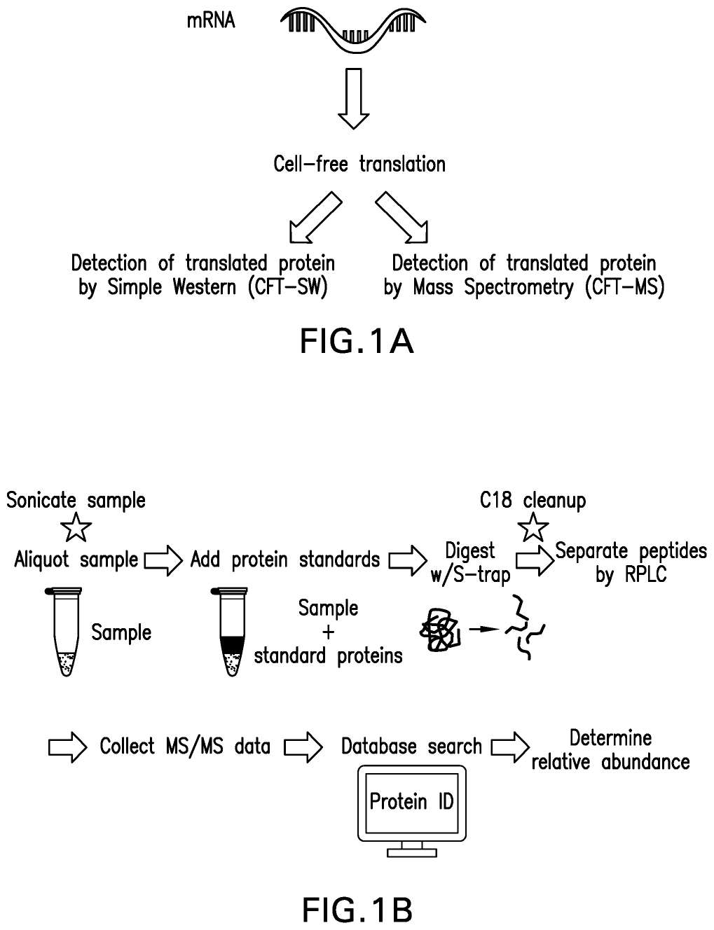

As used herein, “bottom-up proteomics” refers to the approach in which information about the constituent proteins is reconstructed from fragment peptides obtained by proteolytic digestion of proteins and individually identified by mass spectrometry. The various steps of the bottom-up proteomics are outlined in FIG. 1B and described below in Example 1.

It should be noted also that the cell-free or cell-based translation mixture is highly viscous. In this regard, it was observed that compared to more straightforward bottom-up sample preparation workflows found in the art, introducing a viscosity reduction measure such as sonication following cell-free translation—in addition to using a C18 cleanup step—greatly alleviated column clogging. There are many options for C18 cleanup, including, without limitation, using C18 Evotips, when Evosep One LC is used for liquid chromatography, or other C18 tips such as the C18 tips from Pierce (Thermo Fisher, Waltham, MA, USA) when other liquid chromatography systems such as nanoAcquity nano LC (Waters Corporation, Milford, MA, USA) are being used.

As noted above, compared to traditional immunoassay-based detection techniques, using LC-MS/MS as the readout for translated protein provides several advantages. For example, detection of the translated protein by LC-MS/MS eliminates the need for an antibody specifically recognizing the translated protein. Such antibody may not always be available, and time-consuming antibody production and screening may be required to obtain one.

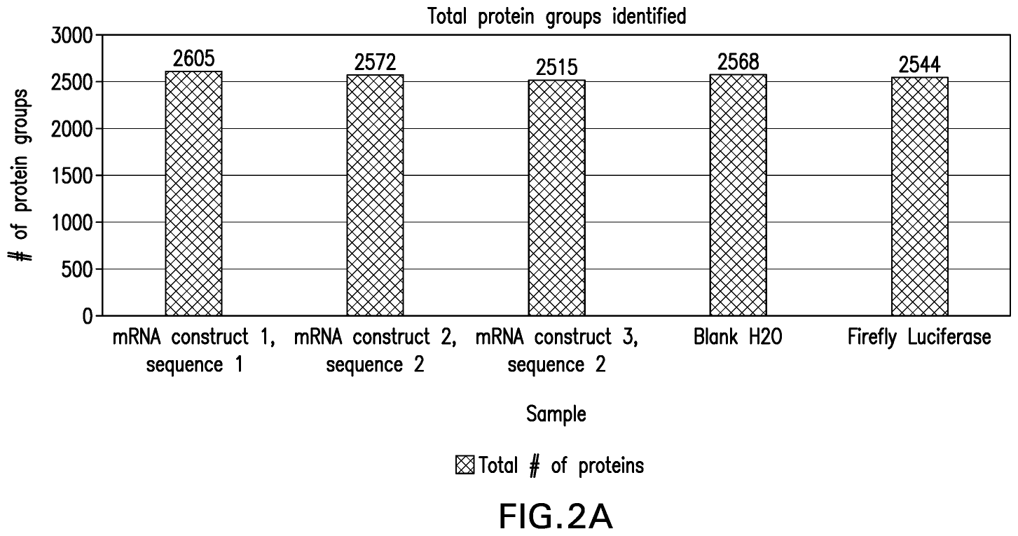

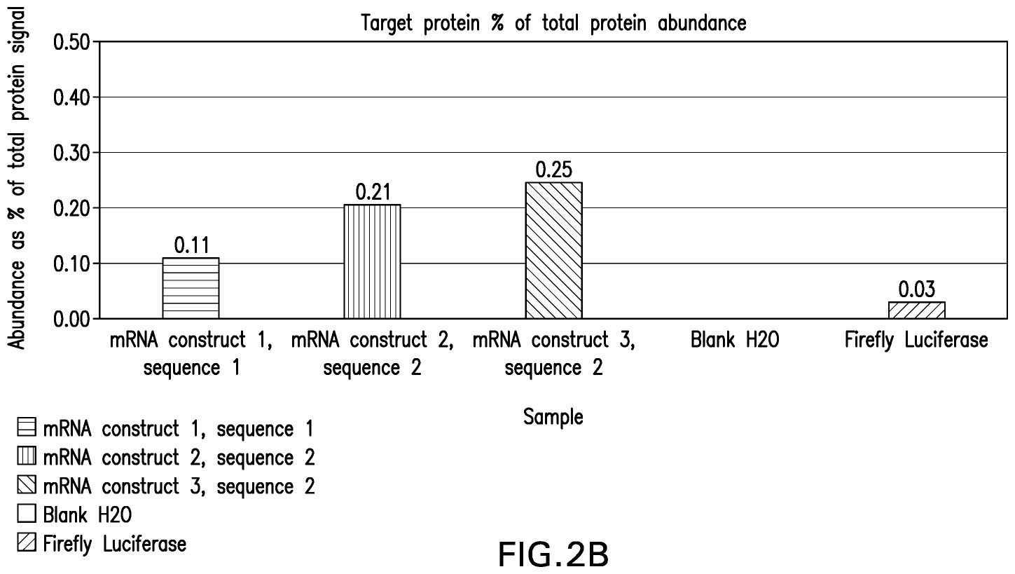

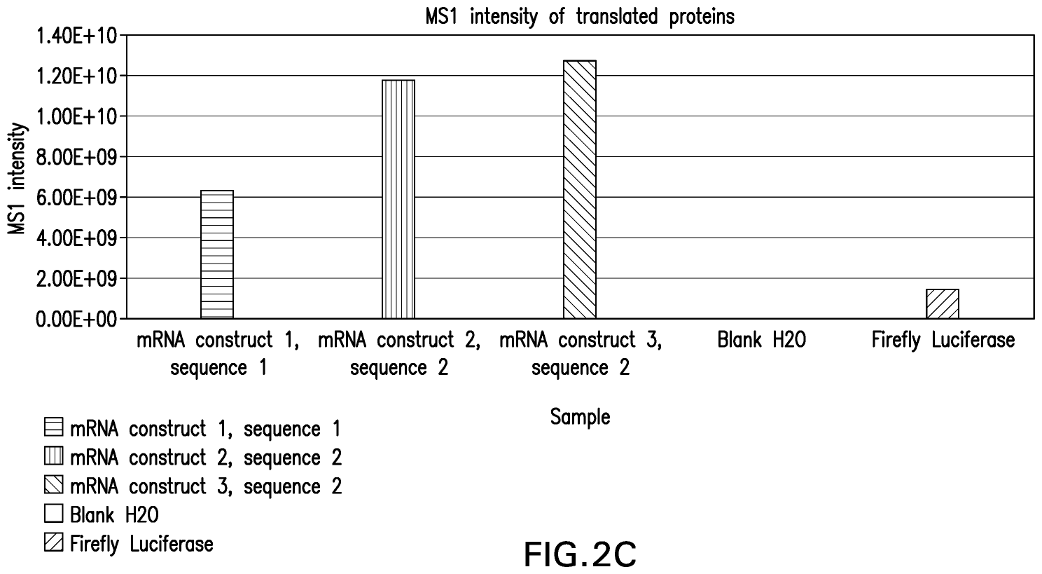

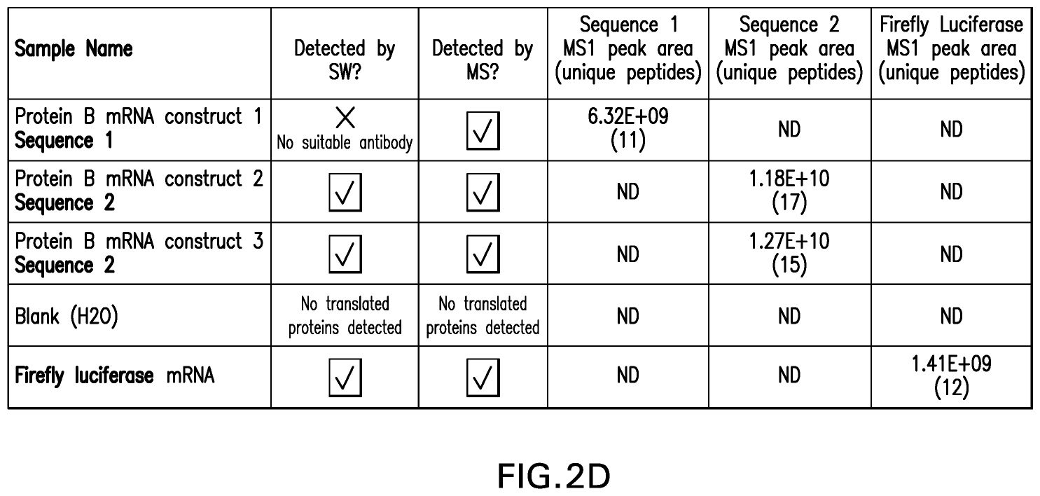

Furthermore, the CFT-MS and CBT-MS methods allow for excellent specificity. That is, even when the translated protein is only a tiny fraction of the total protein content, which includes all the proteins in the cell-free translation system (e.g., less than 0.15%; see FIG. 2B, mRNA construct 1), it is possible to correctly identify the translated protein. This is illustrated by the results of experiments carried out to test the specificity of the method (see Example 3, Table 1, and FIG. 2C). In these experiments, four different mRNAs were translated separately using wheat germ extract as the CFT system. MS1 peaks from each CFT reaction was searched against a wheat proteome database to which the amino acid sequence of protein translated from the mRNA of interest was added.

As used herein, MS1 refers to the parent MS scan in tandem mass spectrometry, which is followed by further scans in which high abundance peaks from MS1 are subjected to further fragmentation and the process is repeated until all candidate peaks of a parent scan are exhausted (Karpievitch Y V et al. Ann Appl Stat. 2010, 4(4), 1797-1823). This process results in a fragmentation pattern for each selected MS1 peptide, which provides detailed information about the chemical makeup of the peptide.

As is known to a person of ordinary skill in the art, protein identification in the context of cell-free or cell-based translation using mass spectrometry involves comparing the peaks produced by mass spectrometry (i.e., MS1 peaks) to the peaks present in a theoretical spectrum generated from peptides produced from enzymatic digestion of all proteins present in the translation system plus the protein of interest (i.e., the protein being translated from the input mRNA). In each of CFT reactions described herein (see Example 3), the protein produced from the translation of a particular mRNA was correctly identified amid over 2500 proteins of the wheat germ extract—even when its abundance was less than 0.15% (see FIG. 2B, first bar from the left).

In addition, the CFT-MS method allows for determination of the amino acid sequence of the translated protein by repeated peptide fragmentation and scanning following the parent scan (MS1), making it possible to confirm the protein identification, and ultimately to confirm the mRNA sequence. This method is described in greater detail in Example 2 using CFT-LC-MS/MS of firefly luciferase protein.

Further, compared to immunoassay-based techniques, the CFT-MS method allows for better quantification of translated proteins since the translated protein is quantified relative to a standard protein(s) which serves as internal standard across different samples. The standard protein(s) is introduced in the CFT sample prior to the enzymatic digestion step. Quantification can be useful in many circumstances, for example, in the identification of an mRNA variant that translates the best from among several variants of that mRNA.

While cell-free translation (to generate proteins from mRNA) or cell-free transcription-translation (to generate proteins from a DNA template) is rapid, cost-effective, and free of common difficulties associated with in vivo or cell-based protein expression systems, the components of the cellular extract used for such cell-free protein expression are challenging to characterize. LC-MS/MS proteomics can be used to characterize and troubleshoot the protein components of these cellular extracts (Hurst, G B et al., Anal. Chem. 2017, 89, 21, 11443-11451). Successful identification of all proteins that make up the ribosome as well as those that are required for transcription and translation can provide assurance regarding the efficacy of translation of an mRNA or that of transcription-translation of a DNA template. It is contemplated herein that the CFT-MS method described herein can be used as an improved tool for characterizing the components of the cellular extracts used in cell-free translation or cell-free transcription-translation. This would require minimal or no additional steps in sample preparation or analysis over those described herein for the characterization of the translated protein from an mRNA of interest.

Furthermore, the information generated using this platform method could be utilized in method development for absolute quantitation mass spectrometry methods, e.g., by multiple-reaction monitoring (MRM) utilizing heavy-labeled peptides or proteins. MRM, also known as SRM (Selective Reaction Monitoring) is a highly specific and sensitive mass spectrometry technique that can selectively quantify compounds within complex mixtures. MRM requires isotope-labeling. A known amount of isotope-labeled authentic standard is mixed with the analyte, the mixture is introduced into mass spectrometer, and the absolute amount of the analyte is calculated from the ratio of ion intensity between the analyte and its standard (Kito, K and Ito, T, Current Genomics, 2008, vol. 9, 263-274). It is contemplated herein that the CFT-MS method described herein can be adapted to generate an MRM method for more sensitive and accurate protein quantitation. Without limitation, applications in which such MRM method would be suited include (1) programs requiring high throughput, (2) programs where the protein of interest and its sequence is known and samples containing the protein need to be analyzed frequently, (3) programs requiring high sensitivity and/or quantitation, and (4) programs that need to be made ready for GMP quality control.

### LC-MSMS Analysis

In the CFT-MS or CBT-MS methods described herein, following LC-MS/MS, the raw data obtained was searched using the application, Proteome Discoverer 2.2 (Thermo Fisher, Waltham, MA, USA). Other equivalent applications that identify proteins from the mass spectra of digested fragments may also be used. The Proteome Discoverer application uses workflows to process and report mass spectrometry data. See Proteome Discoverer User Guide, Software Version 2.2, XCALI-97808 Revision A. June 2017, Thermo Fisher Scientific Inc. As described in the Thermo Fisher Guide, Proteome Discoverer 2.2 compares raw data taken from mass spectrometry with the information obtained from a selected FASTA database and identifies proteins from the mass spectra of digested fragments. It works with peak-finding search engines such as Sequest™ HT and Mascot to process all data types collected from low- and high-mass-accuracy mass spectrometry (MS) instruments. The peak-finding algorithm searches the raw mass spectrometry data and generates a peak list and relative abundances. The peaks represent the fragments of peptides for a given mass and charge. It produces complementary data from a variety of dissociation methods and data-dependent stages of tandem mass spectrometry. It combines, filters, and annotates results from several database search engines and from multiple analysis iterations. The search engines correlate the uninterrupted tandem mass spectra of peptides with databases, such as FASTA.

In the methods described herein, the Sequest™ HT search algorithm was used, and data was searched against the Uniprot Triticum aestivum (wheat) protein database. The expected amino acid sequence of the protein corresponding to the mRNA being translated was added to the database. The search parameters were set to require a minimum of two unique peptides for positive identification of a protein. Relative abundance of each translated protein was calculated using the abundance output (summed MS1 intensity) from the results of the Proteome Discoverer application.

### Embodiments of the CFT-MS or CBT-MS Method

Various aspects of the method for assessing translation efficacy of one or more mRNAs described herein are set forth in greater detail in the numbered embodiments that follow.

Embodiment 1 provides a method for assessing translation efficacy of one or more mRNAs, the method comprising:
(a), translating the one or more mRNAs, wherein the one or more mRNAs are present in a cell lysate (cell-free translation) or within a cell (cell-based translation) thereby obtaining a translation reaction product comprising a protein or proteins translated from the one or more mRNAs;
(b) digesting the translation reaction product with a first proteolytic enzyme, thereby obtaining a first enzymatic digest;
(c) subjecting the first enzymatic digest to liquid chromatography-tandem mass spectrometry (LC-MS/MS), thereby obtaining a first peptide profile; and
(d) analyzing the first peptide profile by detecting peptides derived from the one or more proteins; and
(e) optionally further analyzing the first peptide profile, thereby determining amino acid sequence or sequences of the one or more proteins; and comparing the amino acid sequence or sequences thus obtained to an amino acid sequence or sequences expected from translation of the one or more mRNAs, thereby verifying, nucleotide sequence or sequences of the one or more mRNAs;
thereby assessing the translation efficacy of the one or more mRNAs.

Embodiment 2 provides the method of embodiment 1, wherein the translation is carried out in a cell lysate.

Embodiment 3 provides the method of embodiment 1, wherein the translation is carried out within a cell.

Embodiment 4 provides the method of embodiment 3, wherein the one or more mRNAs are provided in a lipid nanoparticle.

Embodiment 5 provides the method of any of embodiments 1-4, wherein analyzing in step (d) further comprises quantifying peptides derived from the one or more proteins.

Embodiment 6 provides the method of any of embodiments 1-5, and further comprises:
(f) digesting the translation reaction product with a second proteolytic enzyme, thereby obtaining a second enzymatic digest;
(g) subjecting the second enzymatic digest to LC-MS/MS, thereby obtaining a second peptide profile;
(h) analyzing the second peptide profile by detecting peptides derived from the one or more proteins;
(i) further analyzing the second peptide profile, thereby determining amino acid sequence or sequences of the one or more proteins; and comparing the amino acid sequence or sequences thus obtained to an amino acid sequence or sequences expected from translation of the one or more mRNAs, thereby verifying nucleotide sequence or sequences of the one or more mRNAs; and;
(j) combining information from the amino acid sequence or sequences of the one or more proteins obtained in step (e) with that obtained in step (i), thereby assembling an amino acid sequence or amino acid sequences of the one or more proteins; and comparing the assembled amino acid sequence or sequences to an amino acid sequence or sequences expected from translation of the one or more mRNAs, thereby further verifying nucleotide sequence or sequences of the one or more mRNAs: thereby assessing the translation efficacy of the one or more mRNAs.

Embodiment 7 provides the method of embodiment 6, wherein analyzing in step (h) further comprises quantifying peptides derived from the one or more proteins.

Embodiment 8 provides the method of any of embodiments 1-7, wherein assessing translation efficacy of the one or more mRNAs comprises comparing the translation of the one or more mRNAs to the translation of a control mRNA previously determined to have optimal translation.

Embodiment 9 provides the method of embodiment 8, wherein the control mRNA is included in the cell-free translation performed in step (a) and is translated along with the one or more mRNAs.

Embodiment 10 provides the method of embodiment 8, wherein the control mRNA is translated separately using a method according to steps (a)-(d), and optionally according to steps (a) and (f)-(h).

Embodiment 11 provides the method of any of embodiments 1-10, wherein analyzing translation of the one or more mRNAs comprises identifying at least two peptides predicted to be generated by the enzymatic digestion of the one or more proteins translated from the one or more mRNAs.

Embodiment 12 provides the method of any of embodiments 1-11, wherein the MS1 intensity of a peptide in the peptide profile is indicative of the relative abundance of a translated protein from which the peptide is generated.

Embodiment 13 provides the method of any of embodiments 1-12, wherein prior to step (b), the translation reaction product is enriched for proteins by removal of non-protein components using a suspension trapping column, thereby yielding a protein-enriched translation reaction product.

Embodiment 14 provides the method of embodiment 13, wherein step (b) comprises digesting the protein-enriched translation reaction product on the column and, prior to step (c), eluting from the column, peptides resulting from the digestion.

Embodiment 15 provides the method of any of embodiments 6-13, wherein prior to step (f), the translation reaction product is enriched for proteins by removal of non-protein components using a suspension trapping column, thereby yielding a protein-enriched translation reaction product.

Embodiment 16 provides the method of embodiment 15, wherein step (f) comprises digesting the protein-enriched translation reaction product on the column and, prior to step (g), eluting from the column, peptides resulting from the digestion.

Embodiment 17 provides the method of any of embodiments 1-16, further comprising enriching the first enzymatic digest for peptides using a C18 column prior to step (c).

Embodiment 18 provides the method of any of embodiments 6-17, further comprising enriching the second enzymatic digest for peptides using a C18 column prior to step (g).

Embodiment 19 provides the method of any of embodiments 1-18, wherein the LC-MS/MS comprises separating peptides present in the enzymatic digest using reversed-phase liquid chromatography prior to performing mass spectrometry.

Embodiment 20 provides the method of any of embodiments 1-19, wherein the translation reaction product is treated to reduce viscosity prior to enzymatic digestion.

Embodiment 21 provides the method of any of embodiments 1-20, wherein the translation product is sonicated to reduce viscosity.

Embodiment 22 provides the method of any of embodiments 1-21, wherein the first proteolytic enzyme used for digesting the translation reaction product is selected from the group consisting of trypsin, chymotrypsin, Asp-N, Glu-C, Lys-C, Lys-N, elastase, thermolysin, proteinase K, Staphylococcus aureus v8 protease, and pepsin.

Embodiment 23 provides the method of any of embodiments 6-22, wherein the second proteolytic enzyme used for digesting the translation reaction product is selected from the group consisting of trypsin, chymotrypsin, Asp-N, Glu-C, Lys-C, Lys-N, elastase, thermolysin, proteinase K, Staphylococcus aureus v8 protease, and pepsin.

Embodiment 24 provides the method of any of embodiments 6-23, wherein both the first and the second proteolytic enzyme used for digesting the translation reaction product are selected from the group consisting of trypsin, chymotrypsin, Asp-N, Glu-C, Lys-C. Lys-N, elastase, thermolysin, proteinase K. Staphylococcus aureus v8 protease, and pepsin.

Embodiment 25 provides the method of embodiment 23 or embodiment 24, wherein the first proteolytic enzyme is one of trypsin and chymotrypsin and the second proteolytic enzyme is the other of trypsin and chymotrypsin.

Embodiment 26 provides the method of embodiment 23 or embodiment 24, wherein the first proteolytic enzyme is one of trypsin and Lys-C and the second proteolytic enzyme is the other of trypsin and Lys-C.

Embodiment 27 provides the method of any of embodiments 1-26, wherein the cell-free translation is performed using rabbit reticulocyte lysate.

Embodiment 28 provides the method of any of embodiments 1-26, wherein the cell-free translation is performed using wheat germ extract.

Embodiment 29 provides the method of any of embodiments 1-28, wherein at least one known protein is added to the translation product prior to the enzymatic digestion to serve as a reference protein in step (d).

Embodiment 30 provides the method of any of embodiments 6-29, wherein at least one known protein is added to the translation product prior to the enzymatic digestion to serve as a reference protein in step (h).

Embodiment 31 provides the method of embodiment 29 or embodiment 30, wherein the at least one known protein comprises a protein selected from the group consisting of alcohol dehydrogenase, myoglobin, carbonic anhydrase, phosphorylase B, and protein A.

### EXAMPLES

### Example 1: Cell-Free mRNA Translation Process

Materials: Nuclease-free water was purchased from Thermo Fisher Scientific (Waltham, MA, USA). Amino acid mixture, complete (1 mM), potassium acetate (1 M), RNasin Plus RNase inhibitor (40 u/μL), and Wheat Germ Extract were all purchased from Promega (Madison, WI, USA). Sodium citrate (1 mM; pH 6.5±0.1) was purchased from Invitrogen (Waltham, MA, USA). 10× Sample Buffer 2 was purchased from ProteinSimple Instruments (Minneapolis, MN, USA). 20% SDS solution was purchased from EMD Millipore (Burlington, MA, USA). Urea. PROTEIN STANDARDS were purchased from Sigma-Aldrich (St. Louis, MO, USA). Glycine was purchased from J. T. Baker (Phillipsburg, NJ, USA). Dithiothreitol and iodoacetamide were purchased from Thermo Scientific (Waltham, MA. USA). 85% phosphoric acid was purchased from Oakwood Chemical (Estill, SC, USA). LCMS-grade methanol. LCMS-grade 0.1% formic acid in water, and 0.1% formic acid in acetonitrile were purchased from Honeywell (Charlotte, NC, USA). Triethylammonium bicarbonate buffer (1.0 M, pH 8.5) was purchased from Combi-Blocks (San Diego, CA, USA). LCMS-grade water, LCMS-grade acetonitrile, and LCMS-grade formic acid were purchased from Fisher Chemical (Waltham, MA, USA). Suspension trap micros (S-trap) were purchased from Protifi (Farmingdale NY). Evosep One C18 Evotips (EV2001) and analytical column (EV 1106, C18, 15 cm long, 1.9 μm beads, and 150 μm inner diameter) were purchased from Evosep (Odense, Denmark).

Cell-free translation sample preparation: Cell free translation was performed according to guidelines provided by Promega, but with the following changes: (1) The component “Amino Acid Mixture, Minus Methionine, 1 mM” was replaced with “Amino Acid Mixture Complete, 1 mM”. (2) The component [³⁵S]methionine was not used and was replaced with water; (3) Reaction components were mixed prior to adding RNA, and (4) RNA was not heat denatured prior to in vitro translation.

The resulting cell free translation method was performed as follows: Lab bench surface was wiped with RNAseZAP™ wipes followed by 70% IPA (iso propyl alcohol). mRNA, stored at −70° C., was thawed. 40 μL of wheat germ extract, 6.4 μL of amino acid mixture, complete (1 mM), 6.4 μL of potassium acetate (1 M), 1.6 μL of RNasin® Plus RNase inhibitor (40 u/μL), and 17.6 μL of nuclease-free water were combined in a tube. mRNA was then added to the tube. The tubes were flicked to mix the contents and then briefly centrifuged, ensuring no air bubbles were present. The samples were incubated in a heat block at 25° C. for 2 hours. 1.1× Simple Western Sample Buffer was prepared. To quench the reaction, 20 μL of in vitro translation reaction mixture and 180 μL of 1.1× Simple Western Sample Buffer were combined, briefly vortexed, and then immediately stored at −70° C. until samples for mass spectrometry were prepared.

Mass spectrometry sample preparation: 20 μL of sample was combined with 30 μL of LCMS-grade water and sonicated for 5 minutes. Following this, a mixture of alcohol dehydrogenase, myoglobin, carbonic anhydrase, phosphorylase B, and protein A was added to the sample as standards. Next, lysis buffer (10% SDS, 8 M urea, and 100 mM glycine) was added in a 1:1 v:v ratio to the sample. Dithiothreitol was added to a final concentration of 5 mM and the sample incubated at 37° C. for 1 hour. The sample was then removed from the incubator and allowed to cool to room temperature. Next, iodoacetamide was added to a final concentration of 15 mM and the sample incubated at 25° C. for 30 minutes in the dark. Then, a solution of 27.5% phosphoric acid was added to the sample to a final concentration of ˜2.5% phosphoric acid. Next, S-trap binding buffer (100 mM triethylammonium bicarbonate (TEAB) in 90% methanol), at a volume of 7.2× the sample volume at this step, was added to the sample. The final solution was added to the top of an S-trap micro column in 180 μL aliquots and spun at 4,000 g for 30 seconds each time until no sample remained. Following binding of the sample to the S-trap, the S-trap column was washed by adding 150 μL of S-trap binding buffer and spinning at 4,000 g for 30 seconds. The washing step was repeated two additional times for a total of three washes.

Next, 2 μg of trypsin in 20 μL of 50 mM TEAB (triethylammonium bicarbonate) was added to the top of the S-trap column while ensuring that no bubbles formed. The top of the column was wrapped in parafilm, and the S-trap cap was gently placed on top (not screwed on tightly). The S-trap column was incubated at 37° C. overnight with no mixing to ensure that no bubbles formed. Following overnight incubation, 40 μL of 50 mM TEAB was added to the top of each column and the spun at 10.000 g for 1 minute. The flowthrough was kept in the collection tube. Next, 40 μL of 0.2% formic acid in water was added to the top of the S-trap column and spun at 10,000 g for 1 minute. The flowthrough was allowed to combine with the flowthrough from the previous step. Next, 40 μL of 50:50 acetonitrile:water was added to the top of the S-trap column and spun at 10,000 g for 1 minute. The flowthrough was again allowed to combine with the flowthrough from the previous two steps in the collection tube. The collected peptides were then dried using a refrigerated vacuum concentrator (SpeedVac™) and then reconstituted in 20 μL of 0.1% formic acid in water. The reconstituted sample was cleaned up using a disposable C18 trap column (Evotip C18 tip; Evosep, Odense, Denmark). C18 resins significantly improve signal-to-noise ratios and sequence coverage by removing MS-incompatible salts and detergents commonly used in protein or peptide preparation. While Evotip C18 tip was used when the LC being used for LC-MS/MS was Evosep One LC (Evosep, Odense, Denmark), C18 ZipTip (Thermo Fisher Scientific, Waltham, MA, USA) was used when the LC being used for LC-MS/MS was nanoAcquity nanoLC (Waters Corporation. Milford, MA, USA).

Liquid chromatography-mass spectrometry analysis: A Thermo Orbitrap™ Exploris 480 mass spectrometer (Thermo Fisher Scientific, Waltham, MA, USA) combined with a Waters nanoAcquity (Waters, Milford, MA, USA) or an Evosep One (Evosep, Odense, Denmark) liquid chromatography system was used for analysis of samples. Samples were injected, separated by reversed-phase LC, and MS/MS data was collected in a data-dependent manner. When using the Evosep One, the “Extended Method.” running at 88 minutes, allowing for 15 samples per day (“SPD”), was chosen.

Data analysis: Following LC-MS/MS, the data acquired in the LC-MS/MS was searched using Thermo Proteome Discoverer™ application (version 2.2) for peptide and protein mass spectrometry analyses. As noted previously in the specification, the application uses the peak-finding search engine Sequest™ HT to search the raw mass spectrometry data and generates a list of peaks and relative abundances, the peaks representing the fragments of peptides for a given mass and charge. The Uniprot Triticum aestivum (wheat) protein database was searched. To this database, the expected amino acid sequence of the protein corresponding to the mRNA being translated was added. The search parameters required a minimum of two unique peptides for positive identification of a protein. The abundance output (summed MS1 intensity) from the results of the Proteome Discoverer application were used for calculating relative abundance of each translated protein.

### Example 2: CFT-MS Allows Determination of the Sequence of the Translated Protein

This Example shows that in addition to allowing assessment of translation efficacy, the CFT-MS method makes it possible to characterize the amino acid sequence of the translated protein, which allows for confirmation of protein identity, and ultimately the mRNA sequence.

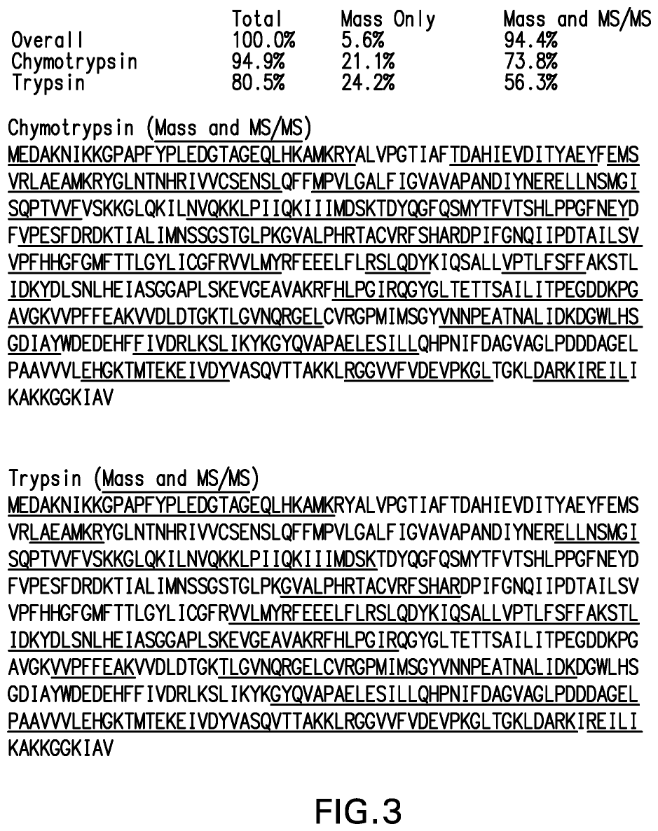

This is highlighted in FIG. 3 which shows an example of the sequence coverage map of the firefly luciferase (fLuc) protein generated from CFT of the fLuc mRNA. When only trypsin was used for digestion, 56.3% sequence coverage of the fLuc protein was obtained by MS2 (FIG. 3, Mass and MS/MS) only and 80.5% sequence coverage was obtained by MS1+MS2 (MS+(MS and MS/MS)). When chymotrypsin was used for digestion, 73.8% sequence coverage was obtained by MS2 (Mass and MS/MS) only and 94.9% sequence coverage was obtained by MS1+MS2 (MS+(MS and MS/MS)). Combining the two analyses, 100% sequence coverage was obtained by MS1+MS2 (MS+(MS and MS/MS)). This example demonstrates the potential of the CFT-MS method to not only provide identification and relative abundance of the translated protein, but also the sequence of the protein.

### Example 3: CFT-MS Permits High Specificity of Detection

The CFT-MS method allows for a high degree of specificity. Even when the translated protein is but only a tiny fraction of the total protein—the total protein including all proteins present in the cell-free translation system—the translated protein can be correctly identified. This is shown by the results of the experiments carried out to test the specificity of the method (see FIGS. 2A-2D).

Five separate cell-free translation reactions were carried out, four each with a particular mRNA construct and one control with no mRNA. See FIG. 2A, in which, the reactions, from left to right, are: (1) mRNA construct 1 (having amino acid sequence 1), (2) mRNA construct 2 (having amino acid sequence 2), (3) mRNA construct 3 (having amino acid sequence 2), (4) Water (control, with no mRNA), and (5) firefly luciferase mRNA construct. mRNA construct 1 encodes the protein, Noro GI.1, and mRNA constructs 2 and 3 encode the closely related protein, Noro GII.4. mRNA constructs 2 and 3 use different codons but produce proteins having the same amino acid sequence. The firefly luciferase mRNA construct was used as a control for the translation reaction.

A similar and high number (over 2500) of protein groups were identified in each case, and except in the control, translated protein was detected even though it constituted no more than 0.25% and as little as 0.11% of the total protein content of the reaction (FIG. 2B). Furthermore, it was observed that in each reaction, the MS1 peak corresponded to the peak expected from the protein translated in that sample (FIG. 2C), and not to the peak expected of the closely related protein. For example, in the sample in which mRNA construct 1 (encoding Noro GI.1 protein) was translated, only Noro GI.1 protein was identified, not Noro GII.4 (encoded by mRNA construct 2 or 3).

### Example 4: CFT-MS can Provide Relative Quantification of Translated Proteins

The LC-MS/MS method described herein provides relative quantification of the translated protein with a standard protein(s) serving as an internal standard(s) (e.g., BSA) across different samples (translation reactions). This is an advantage compared to immunoassay-based methods, which are not fully quantitative.

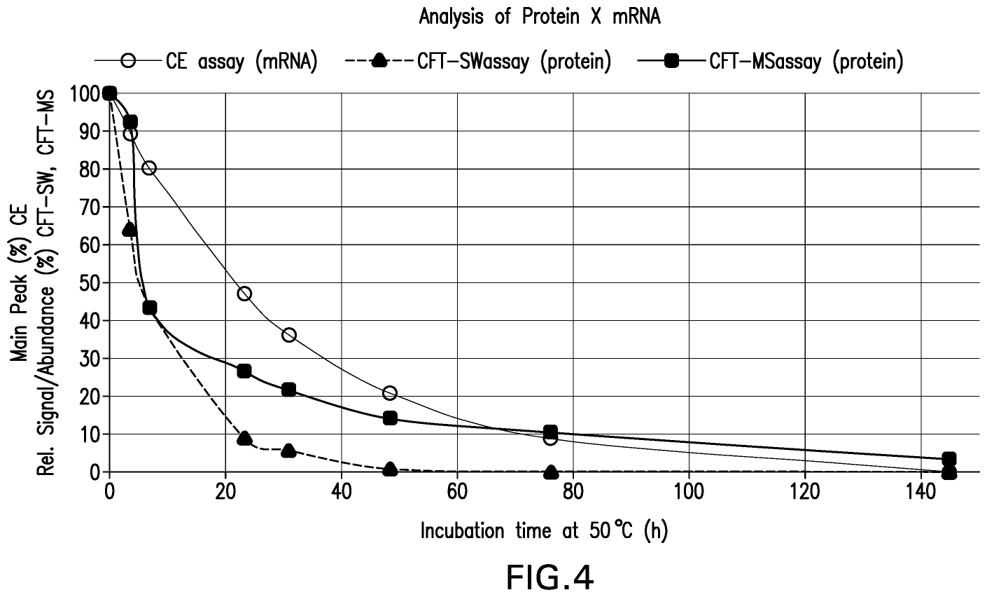

The ability to obtain relative quantification is highly useful, for example, when testing different mRNA preparations to determine if there has been degradation. In this Example, mRNA for a test protein (Protein X) was thermally degraded by incubating at 50° C. for different lengths of time and translation was assessed for each incubation period. Capillary electrophoresis (CE) analysis carried out showed that the mRNA was indeed degraded (FIG. 4; filled circle). CFT was carried out with each mRNA sample and the translated protein was identified by SW or by MS. As expected, a trend like that of mRNA degradation (CE analysis) was observed for the relative abundance of the translated protein using both SW and by MS (FIG. 4). CFT-MS was found to offer as good or better sensitivity compared to CFT followed by SW.

### Example 5: CFT-MS Allows Reliable Detection and Determination of Relative Abundances of Proteins Translated from Multiple mRNAs in the Same Sample

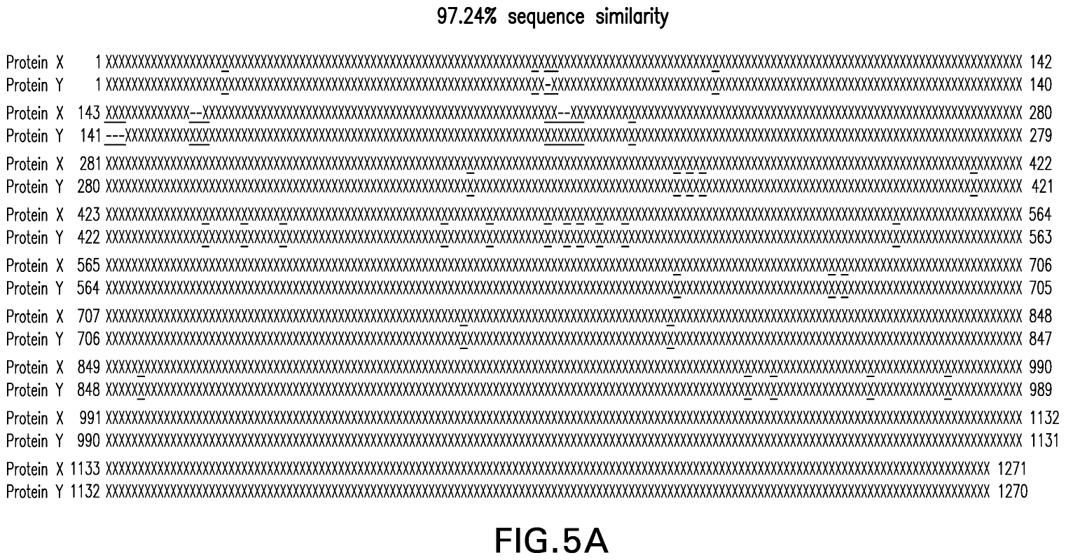

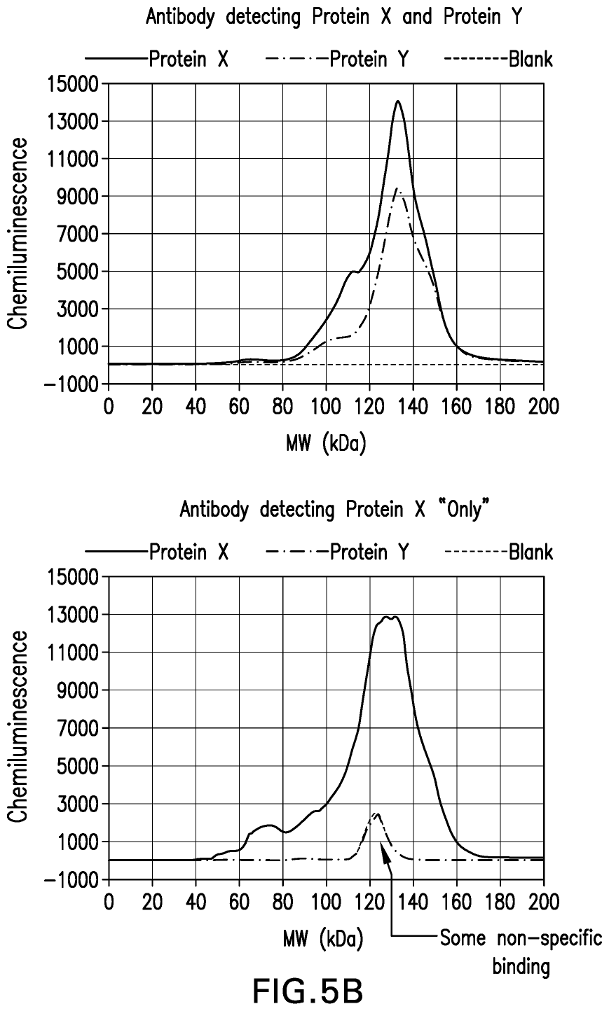

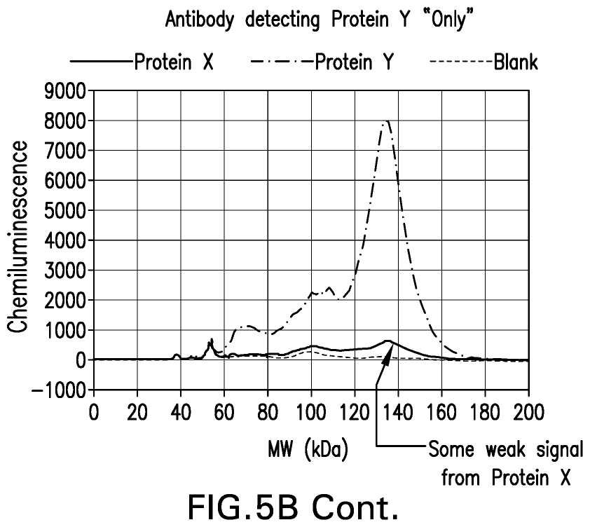

At times, there may be a need to translate a mixture of mRNAs rather than a single mRNA in the same translation reaction. An example is a multivalent mRNA vaccine (which comprises multiple mRNAs). In such vaccines, it is important to be able to determine whether each individual mRNA is translated with expected efficacy. Also, in multivalent vaccines, it is common for the sequences of the individual translated proteins to overlap significantly. This is exemplified by two test proteins X and Y (FIG. 5A). These proteins share 97.24% sequence identity. An SW analysis of each of these proteins using antibodies is shown in FIG. 5B. In this Figure, results obtained using an antibody that detects both Protein X and Protein Y is shown on the left, that obtained using an antibody that detects only Protein X is shown in the middle, and that using only Protein Y is shown on the right. The anti-Protein X antibody was found to also exhibit non-specific binding (see arrow mark in the middle graph) and anti-Protein Y antibody was found to weakly recognize Protein X, i.e., exhibit cross reactivity (see arrow mark in the graph on the right). Both antibody cross reactivity and non-specific binding can interfere with calculation of the relative abundances of translated proteins leading to ambiguity in determining translation efficacy.

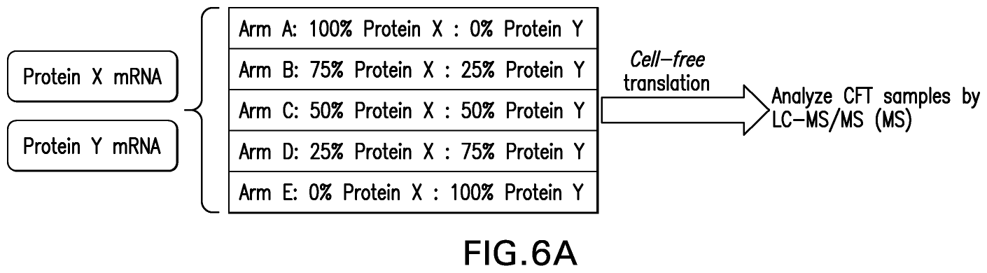

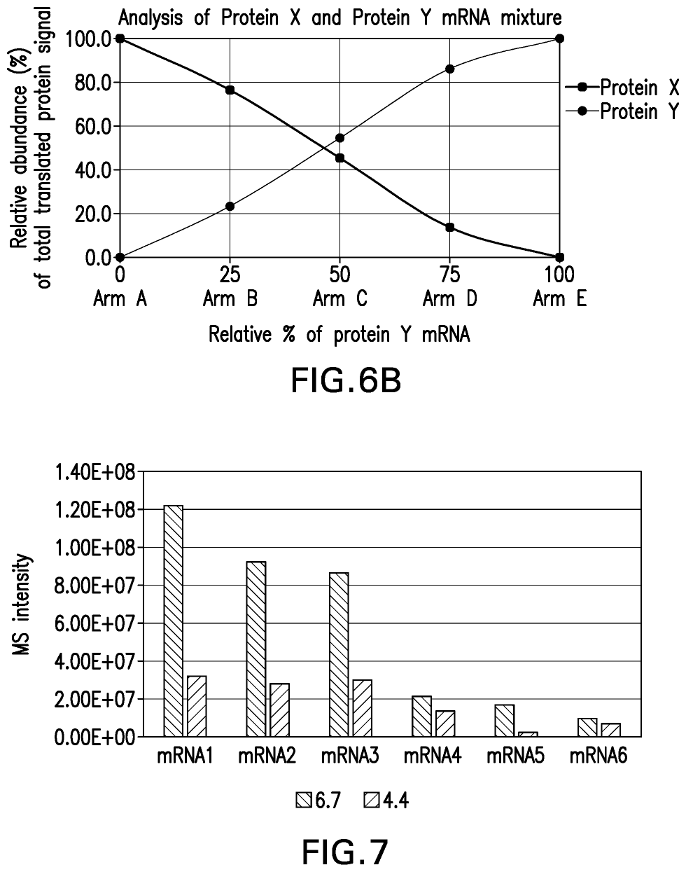

To overcome this problem, an experiment was designed to see if relative abundances could be reliably obtained using CFT-MS. In this experiment. Protein X and Protein Y mRNAs were combined in varying ratios in different mixtures and CFT carried out with each mixture (see FIG. 6A). The results of relative abundances (%) of each of Protein X and Protein Y obtained from MS analysis of these samples are shown in FIG. 6B. The results demonstrate that CFT-MS can accurately characterize proteins translated from mRNA mixtures even when there is high sequence homology between the sequences of the proteins.

Thus, in contrast to an immunoassay-based technique, which not only requires separate analysis of each protein translated from a mixture of mRNAs, and the results may be inaccurate due to antibody cross-reactivity and/or non-specific binding, CFT-MS can reliably detect multiple translated proteins and provide their relative abundances in a single LC-MS run.

### Example 6: Cell-Based Translation (CBT-MS)

LC-MS/MS, used in CFT-MS assay described in the Examples above, also allows reliable determination of relative abundances of proteins translated from mRNA within a cell (Cell-based translation; “CBT-MS”). As such, LC-MS/MS can be used for determining potency and identity of an mRNA in a drug product (DP) formulation, such as a lipid nanoparticle (LNP) after transfer of the LNP into cells.

Conventional assays for determining mRNA DP potency use image-based techniques (e.g., immunofluorescence), in which the protein translated from the mRNA DP upon transfection of cells with the DP is detected using a suitable antibody. This requires a suitable antibody (i.e., antibody with high affinity and minimal cross-reactivity). In contrast, as shown below, potency of either a single mRNA DP or a multi-valent mRNA DP can be determined through relative quantification of the translated proteins in cells using LC-MS/MS without using an antibody.

A hexavalent mRNA DP comprising mRNAs 1 to 6, each at a dose of 6.7 or 4.4 ng, was incubated with Huh7 cells (85-95% confluency) in a 96 wells plate for 18 h at 37° C. After incubation, the supernatant was removed, and the cell monolayer gently washed three times with about 200 μl PBS. The cells were lysed with 100 μl of cell lysis buffer in the EasyPep™ MS Sample Prep kit (Thermo Fisher Scientific, Waltham, MA). After mixing vigorously (10-15×), the collected cell lysate containing translated proteins was digested using the S-trap digestion as described above, and the digest was analyzed by LC-MS/MS. The relative quantification of the translated proteins in cells was determined using the peak area of the identified protein after database search of the MS files. As shown in FIG. 7, the translated proteins from all 6 mRNAs in the DP co-formulated as LNP could be identified with confidence (>=2 unique peptides) and quantified. The cells transfected with more mRNA (6.7 ng) produced more proteins than those from the lower doses (4.4 ng).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Various structural elements of the different embodiments and various disclosed method steps may be utilized in various combinations and permutations, and all such variants are to be considered forms of the invention. Scope of the invention is thus indicated by the appended claims rather than by the foregoing description, and all changes that come within the meaning and range of equivalency of the claims are intended to be embraced therein.

1. A method for assessing translation efficacy of one or more mRNAs, the method comprising:
(a) translating the one or more mRNAs, wherein the one or more mRNAs are present in a cell lysate (cell-free translation) or within a cell (cell-based translation) thereby obtaining a translation reaction product comprising a protein or proteins translated from the one or more mRNAs;
(b) digesting the translation reaction product with a first proteolytic enzyme, thereby obtaining a first enzymatic digest;
(c) subjecting the first enzymatic digest to liquid chromatography-tandem mass spectrometry (LC-MS/MS), thereby obtaining a first peptide profile; and
(d) analyzing the first peptide profile by detecting peptides derived from the one or more proteins; and
(e) optionally further analyzing the first peptide profile, thereby determining amino acid sequence or sequences of the one or more proteins; and comparing the amino acid sequence or sequences thus obtained to an amino acid sequence or sequences expected from translation of the one or more mRNAs, thereby verifying, nucleotide sequence or sequences of the one or more mRNAs; thereby assessing the translation efficacy of the one or more mRNAs.

2. The method of claim 1, wherein the translation is carried out in a cell lysate.

3. The method of claim 1, wherein the translation is carried out within a cell.

4. The method of claim 3, wherein the one or more mRNAs are provided in a lipid nanoparticle.

5. The method of claim 1, wherein analyzing in step (d) further comprises quantifying peptides derived from the one or more proteins.

6. The method of claim 1, further comprising:
(f) digesting the translation reaction product with a second proteolytic enzyme, thereby obtaining a second enzymatic digest;
(g) subjecting the second enzymatic digest to LC-MS/MS, thereby obtaining a second peptide profile;
(h) analyzing the second peptide profile by detecting peptides derived from the one or more proteins;
(i) further analyzing the second peptide profile, thereby determining amino acid sequence or sequences of the one or more proteins; and comparing the amino acid sequence or sequences thus obtained to an amino acid sequence or sequences expected from translation of the one or more mRNAs, thereby verifying nucleotide sequence or sequences of the one or more mRNAs; and
(j) combining information from the amino acid sequence or sequences of the one or more proteins obtained in step (e) with that obtained in step (i), thereby assembling an amino acid sequence or amino acid sequences of the one or more proteins; and comparing the assembled amino acid sequence or sequences to an amino acid sequence or sequences expected from translation of the one or more mRNAs, thereby further verifying nucleotide sequence or sequences of the one or more mRNAs; thereby assessing the translation efficacy of the one or more mRNAs.

7. The method of claim 6, wherein analyzing in step (h) further comprises quantifying peptides derived from the one or more proteins.

8. The method of claim 1, wherein assessing translation efficacy of the one or more mRNAs comprises comparing the translation of the one or more mRNAs to the translation of a control mRNA previously determined to have optimal translation.

9. The method of claim 8, wherein the control mRNA is included in the cell-free translation or the cell-based translation performed in step (a) and is translated along with the one or more mRNAs.

10. The method of claim 8, wherein the control mRNA is translated separately using a method according to steps (a)-(d), and optionally according to steps (a) and (f)-(h).

11. The method of claim 1, wherein analyzing translation of the one or more mRNAs comprises identifying at least two peptides predicted to be generated by the enzymatic digestion of the one or more proteins translated from the one or more mRNAs.

12. The method of claim 1, wherein the MS1 intensity of a peptide in the peptide profile is indicative of the relative abundance of a translated protein from which the peptide is generated.

13. The method of claim 1, wherein prior to step (b), the translation reaction product is enriched for proteins by removal of non-protein components using a suspension trapping column, thereby yielding a protein-enriched translation reaction product.

14. The method of claim 13, wherein step (b) comprises digesting the protein-enriched translation reaction product on the column and, prior to step (c), eluting from the column, peptides resulting from the digestion.

15. The method of claim 6, wherein prior to step (f), the translation reaction product is enriched for proteins by removal of non-protein components using a suspension trapping column, thereby yielding a protein-enriched translation reaction product.

16. The method of claim 15, wherein step (f) comprises digesting the protein-enriched translation reaction product on the column and, prior to step (g), eluting from the column, peptides resulting from the digestion.

17. The method of claim 1, further comprising enriching the first enzymatic digest for peptides using a C18 column prior to step (c).

18. The method of claim 6, further comprising enriching the second enzymatic digest for peptides using a C18 column prior to step (g).

19. The method of claim 1, wherein the LC-MS/MS comprises separating peptides present in the enzymatic digest using reversed-phase liquid chromatography prior to performing mass spectrometry.

20. The method of claim 1, wherein the translation reaction product is treated to reduce viscosity prior to enzymatic digestion.

21. The method of claim 1, wherein the translation product is sonicated to reduce viscosity.

22. The method of claim 1, wherein the first proteolytic enzyme used for digesting the translation reaction product is selected from the group consisting of trypsin, chymotrypsin, Asp-N, Glu-C, Lys-C, Lys-N, elastase, thermolysin, proteinase K, Staphylococcus aureus v8 protease, and pepsin.

23. The method of claim 6, wherein the second proteolytic enzyme used for digesting the translation reaction product is selected from the group consisting of trypsin, chymotrypsin, Asp-N, Glu-C, Lys-C, Lys-N, elastase, thermolysin, proteinase K, Staphylococcus aureus v8 protease, and pepsin.

24. (canceled)

25. The method of claim 23, wherein (a) the first proteolytic enzyme is one of trypsin and chymotrypsin and the second proteolytic enzyme is the other of trypsin and chymotrypsin; or the first proteolytic enzyme is one of trypsin and Lys-C and the second proteolytic enzyme is the other of trypsin and Lys-C.

26. (canceled)

27. The method of claim 1, wherein the cell-free translation is performed using rabbit reticulocyte lysate or wheat germ extract.

28. (canceled)

29. The method of claim 1, wherein at least one known protein is added to the translation product prior to the enzymatic digestion to serve as a reference protein in step (d).

30. The method of claim 6, wherein at least one known protein is added to the translation product prior to the enzymatic digestion to serve as a reference protein in step (h).

31. (canceled)


SEQUENCE LISTING (1 sequence)

SEQ ID NO: 1 - protein, 550 aa, synthetic construct
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV